(19) United States
(12) Patent Application Publication
(10) Pub. No.: US 2015/0125526 A1
(43) Pub. Date: May 7, 2015

(54) Use of Cysteamine and Derivatives Thereof to Treat Mitochondrial Diseases

(71) Applicant: Raptor Pharmaceuticals Inc., Novato, CA (US)

(72) Inventors: Patrice Rioux, San Jose, CA (US); Todd C. Zankel, San Francisco, CA (US)

(21) Appl. No.: 14/534,887

(22) Filed: Nov. 6, 2014

Related U.S. Application Data
(60) Provisional application No. 61/900,772, filed on Nov. 6, 2013.

(51) Int. Cl. A61K 31/145; A61K 45/06; A61K 9/50

(52) U.S. Cl. CPC A61K 31/145; A61K 9/5005; A61K 45/06
USPC 424/474; 514/665

(57) ABSTRACT

The present disclosure is directed to methods for treating inherited or acquired mitochondrial disease using a cysteamine product, e.g., cysteamine or cystamine or derivatives thereof.

The present application claims the priority benefit of U.S. Provisional Patent Application No. 61/900,772, filed Nov. 6, 2013, hereby incorporated by reference in its entirety.

### FIELD OF THE INVENTION

The present invention relates to the use of cysteamine or cystamine or derivatives thereof to treat inherited or acquired mitochondrial disorders.

### BACKGROUND

Mitochondria are organelles located within most eukaryotic cells and are responsible for a variety of metabolic transformations and regulatory events, including synthesis and regulation of energy supply. Mitochondria are involved in multiple biological pathways, including oxidative ATP production and synthesis of iron-sulphur clusters, heme, amino acids, steroid hormones and neurotransmitters, regulation of cytoplasmic calcium levels and key events in apoptosis (Tyynismaa et al., EMBO Rep. 10:137-43, 2009).

Adenosine triphosphate (ATP) is the major biochemical mediator of energy transfer and is primarily synthesized by the oxidative phosphorylation chemical pathway. In oxidative phosphorylation (OXPHOS) electrons are transferred from electron donors to electron acceptors such as oxygen, in redox reactions. In eukaryotes, redox reactions are carried out by a series of five related protein complexes within mitochondria, called electron transport chains. There are four basic stages in OXPHOS that include: oxidation of food substances to reducing equivalents such as NAD(P)H; sequential reduction and oxidation of electron transport complexes I, II, III and IV resulting in proton pumping to create an electrochemical potential; reduction of molecular oxygen to generate water; and coupling of the generated electrochemical potential at complex V to the phosphorylation of ADP to generate ATP. These events form the basis of respiration. Additionally, oxidative phosphorylation produces reactive oxygen species (ROS) such as superoxide and hydrogen peroxide, which lead to propagation of free radicals that damage cells and contribute to disease and, possibly, aging (senescence). Impairment of the energy regulation system and ATP synthesis by mitochondria can lead to severe outcomes in affected individuals.

Most of the known mitochondrial disorders are caused primarily by a dysfunctional respiratory chain, often due to inherited or acquired mutations in mitochondrial DNA (mtDNA). The clinical manifestations of mtDNA disorders are extremely heterogeneous due to the complexity of mitochondrial genetics and biochemistry, and include lesions of single tissues or structures (e.g., optic nerve in Leber's hereditary optic neuropathy (LHON)), to widespread lesions in myopathies, encephalomyopathies, cardiopathies, or complex multisystem syndromes. Onset of mitochondrial disorders can range from neonatal to adult life (Zeviani et al., Brain 127:2153-2172, 2004). Adult patients often show signs of myopathy associated with variable involvement of the CNS (ataxia, hearing loss, seizures, polyneuropathy, pigmentary retinopathy and movement disorders). In certain instances, only muscle weakness and/or wasting with exercise intolerance is observed (Zeviani, supra). The most common morphological finding in mitochondrial disorders is perhaps the transformation of scattered muscle fibers into ‘ragged red fibers’ (RRFs), which are characterized by the accumulation of abnormal mitochondria under the sarcolemmal membrane.

Cysteamine (HS—CH₂—CH₂—NH₂) is a small sulfhydryl compound that is able to cross cell membranes easily due to its small size. Cysteamine plays a role in formation of the protein glutathione (GSH) precursor, and is currently FDA approved for use in the treatment of cystinosis, an intra-lysosomal cystine storage disorder. In cystinosis, cysteamine acts by converting cystine to cysteine and cysteine-cysteamine mixed disulfide, which are then both able to leave the lysosome through the cysteine and lysine transporters respectively (Gahl et al., N Engl J Med 347(2):111-21, 2002). Within the cytosol the mixed disulfide can be reduced by its reaction with glutathione and the cysteine released can be used for further GSH synthesis. Treatment with cysteamine has been shown to result in lowering of intracellular cystine levels in circulating leukocytes (Dohil et al., J. Pediatr 148(6):764-9, 2006).

Cysteamine is also discussed in Prescott et al., (Lancet 2(7778):652, 1979); Prescott et al., (Br Med J 1(6116):856-7, 1978); Mitchell et al., (Clin Pharmacol Ther 16(4):676-84, 1974); de Ferreyra et al., (Toxicol Appl Pharmacol. 48(2):221-8, 1979); and Qiu et al., (World J Gastroenterol. 13:4328-32, 2007). Unfortunately, the sustained concentrations of cysteamine necessary for therapeutic effect are difficult to maintain due to rapid metabolism and clearance of cysteamine from the body, with nearly all administered cysteamine converted to taurine in a matter of hours. These difficulties are transferred to patients in the form of high dosing levels and frequencies, with all of the consequent unpleasant side effects associated with cysteamine (e.g., gastrointestinal distress and body odor). See the package insert for CYSTAGON® (cysteamine bitartrate). International Publication No. WO 2007/079670 and U.S. Pat. Nos. 8,026,2854 and 8,129,433 disclose enterically coated cysteamine products and a method of reducing dosing frequency of cysteamine.

Cysteamine is addressed in International Patent Application Nos. WO 2009/070781, and WO 2007/089670, and U.S. Patent Publication Nos. 20110070272, 20090048154, and 20050245433.

### SUMMARY

The present disclosure provides a method of treating a subject suffering from an inherited or acquired mitochondrial disorder comprising administering a therapeutically effective amount of a cysteamine product, e.g., cysteamine or cystamine or derivatives thereof. It is contemplated that administration of the cysteamine product increases levels of free thiols in mitochondrial disease patients, which can improve the detrimental effects of respiratory chain dysfunction in patients. It is understood that such inherited or acquired mitochondrial disorders are due to inherited or acquired mutations in mitochondrial DNA or nuclear DNA used in mitochondria activity.

In various embodiments, the disclosure provides a method of treating a subject suffering from an inherited or acquired mitochondrial disease or disorder comprising administering a cysteamine product or composition, e.g., cysteamine or derivative thereof or cystamine or derivative thereof.

In various embodiments, the mitochondrial disease or disorder is selected from the group consisting of Friedreich's Ataxia, Leber's hereditary optic neuropathy, myoclonic epilepsy and ragged-red fibers (MERRF), Mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes, and MEGDEL syndrome (3-methylglutaconic aciduria type IV with sensorineural deafness, encephalopathy and Leigh-like syndrome. Inherited or acquired mitochondrial diseases contemplated herein exclude diseases caused by CAG repeat expansion in protein-coding portions of non-mitochondrial genes (e.g., Huntington's disease) as well as diseases that may include somatic mutations of mitochondrial DNA due to aging (e.g., Parkinson's disease, Alzheimer's disease).

In various embodiments, the inherited mitochondrial disorder is Friedreich's Ataxia.

In various embodiments, the inherited mitochondrial disorder is Leigh's syndrome. In some embodiments, the Leigh's syndrome patient has a POLG mutation. The disclosure contemplates treating a population of patients having a POLG mutation.

In various embodiments, the total daily dose of cysteamine product (e.g., cysteamine or derivative thereof or cystamine or derivative thereof) is about 0.5-4.0 g/m². Additional doses and dose regimens contemplated herein are described further in the Detailed Description. In various embodiments, the cysteamine product is administered at a frequency of 4 or less times per day (e.g., one, two or three times per day). In various embodiments, the cysteamine product is administered twice daily.

In various embodiments, the cysteamine product is a delayed or controlled release dosage form that provides increased delivery of the cysteamine or cysteamine derivative to the small intestine. In various embodiments, the delayed or controlled release dosage form comprises an enteric coating that releases the cysteamine product when the cysteamine reaches the small intestine or a region of the gastrointestinal tract of a subject in which the pH is greater than about pH 4.5. For example, the coating can be selected from the group consisting of polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters. The composition can be administered orally or parenterally.

In various embodiments, the subject has decreased thiol levels compared to a non-affected subject.

In various embodiments, the administering results in improvement in mitochondrial activity markers compared to levels before administration of the cysteamine composition. Exemplary mitochondrial activity markers include, but are not limited to, free thiol levels, glutathione (GSH), reduced glutathione (GSSH), total glutathione, advanced oxidation protein products (AOPP), ferric reducing antioxidant power (FRAP), lactic acid, pyruvic acid, lactate/pyruvate ratios, phosphocreatine, NADH(NADH+H⁺) or NADPH(NADPH+H⁺), NAD or NADP levels, ATP, anaerobic threshold, reduced coenzyme Q, oxidized coenzyme Q; total coenzyme Q, oxidized cytochrome C, reduced cytochrome C, oxidized cytochrome C/reduced cytochrome C ratio, acetoacetate, β-hydroxy butyrate, acetoacetate/β-hydroxy butyrate ratio, 8-hydroxy-2′-deoxyguanosine (8-OHdG), levels of reactive oxygen species, levels of oxygen consumption (VO2), levels of carbon dioxide output (VCO2), and respiratory quotient (VCO2/VO2).

In various embodiments, the administering results in increased thiol levels compared to levels before administration of the cysteamine product.

In various embodiments, the administering results in improved results in the Newcastle Paediatric Mitochondrial Disease Scale and Barry Albright Dystonia Scale compared to levels before administration of the cysteamine or derivative thereof or cystamine or derivative thereof.

In various embodiments, the cysteamine product is formulated in a tablet or capsule which is enterically coated.

In various embodiments, the cysteamine product is administered parenterally. In various embodiments, the cysteamine product is administered orally.

In various embodiments, the cysteamine product further comprises a pharmaceutically acceptable carrier. It is further contemplated that the cysteamine product is formulated as a sterile pharmaceutical composition.

In various embodiments, the inherited mitochondrial disorder is Friedreich's ataxia. In various embodiments, the inherited mitochondrial disorder is Leber's hereditary optic neuropathy. In various embodiments, the cysteamine product is administered topically in the eye.

In various embodiments, the disclosure provides that a cysteamine product or composition is administered with a second agent useful to treat inherited or acquired mitochondrial diseases or disorders. Exemplary second agents include, but are not limited to, coenzyme Q10, coenzyme Q10 analogs, idebenone, decylubiquinone, Epi-743, resveratrol and analogs thereof, arginine, vitamin E, tocopherol, MitoQ, glutathione peroxidase mimetics, levo-carnitine, acetyl-L-carnitine, dichloroacetate, dimethylglycine, and lipoic acid.

In various embodiments, the subject is a child or adolescent.

In one aspect, the methods of the disclosure also include use of a cysteamine product in preparation of a medicament for treatment an inherited or acquired mitochondrial disease, and use of a cysteamine product in preparation of a medicament for administration in combination with a second agent for treating an inherited or acquired mitochondrial disease. Also included is use of a second agent for treating an inherited or acquired mitochondrial disease in preparation of a medicament for administration in combination with a cysteamine product. Further provided are kits comprising a cysteamine product for treatment of an inherited or acquired mitochondrial disease, optionally with a second agent for treating an inherited or acquired mitochondrial disease, and instructions for use.

### DETAILED DESCRIPTION

The present disclosure relates, in general, to methods of treating inherited or acquired mitochondrial disorders using a cysteamine product, e.g., cysteamine or cystamine or derivatives thereof. It is contemplated that administration of a cysteamine product to a subject suffering from a mitochondrial disease or disorder, especially those in which decreased levels of free thiols are detected, will increase glutathione production and decrease the levels of free radical byproducts that result from oxidative phosphorylation in the mitochondria.

### DEFINITIONS

As used herein and in the appended claims, the singular forms “a,” “and,” and “the” include plural referents unless the context clearly dictates otherwise. Thus, for example, reference to “a derivative” includes a plurality of such derivatives and reference to “a patient” includes reference to one or more patients and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and products, the exemplary methods, devices and materials are described herein.

The documents discussed above and throughout the text are provided solely for their disclosure prior to the filing date of the present application. Nothing herein is to be construed as an admission that the inventors are not entitled to antedate such disclosure by virtue of prior disclosure. Each document is incorporated by reference in its entirety with particular attention to the disclosure for which it is cited.

The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

As used herein “an inherited or acquired mitochondrial disease” refers to a disease of the mitochondria resulting from a mutation in mitochondrial DNA or in nuclear DNA that effects mitochondrial activity. Exemplary inherited or acquired mitochondrial diseases, include, but are not limited to, Friedreich's Ataxia, Leber's hereditary optic neuropathy, myoclonic epilepsy and ragged-red fibers, Mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes and MEGDEL syndrome (3-methylglutaconic aciduria type IV with sensorineural deafness, encephalopathy and Leigh-like syndrome). Inherited or acquired mitochondrial diseases contemplated herein exclude diseases caused by CAG repeat expansion in protein-coding portions of non-mitochondrial genes (e.g. Huntington's disease) as well as diseases that may include somatic mutations of mitochondrial DNA due to aging (e.g., Parkinson's disease, Alzheimer's disease).

As used herein, a “therapeutically effective amount” or “effective amount” refers to that amount of a cysteamine product sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective dose refers to that ingredient alone. When referring to a combination, a therapeutically effective dose refers to combined amounts of the active ingredients that result in the therapeutic effect, whether administered in combination, including serially or simultaneously. In various embodiments, a therapeutically effective amount of the cysteamine product ameliorates symptoms, including but not limited to, lactic acidosis, muscle weakness, reduced motor function, neurological damage or abnormalities, brain damage or abnormalities, cerebellar dysfunction, diabetes or hyperglycemia, reduced cardiac function or damage, reduced kidney function or damage, reduced liver function or damage.

“Treatment” refers to prophylactic treatment or therapeutic treatment. In certain embodiments, “treatment” refers to administration of a compound or composition to a subject for therapeutic or prophylactic purposes.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

“Diagnostic” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

As used herein an “improvement in mitochondrial activity markers” refers to a beneficial change in (bio)markers of the mitochondria subsequent to administration of a cysteamine product or composition compared to levels before administration of the cysteamine product or composition. Mitochondrial activity markers, or mitochondrial marker or biomarker, include proteins or metabolites involved in cellular respiration detectable in the mitochondria, including but not limited to, free thiol levels, glutathione (GSH), reduced glutathione (GSSH), total glutathione, advanced oxidation protein products (AOPP), ferric reducing antioxidant power (FRAP), lactic acid, pyruvic acid, lactate/pyruvate ratios, phosphocreatine, NADH(NADH+H⁺) or NADPH(NADPH+H⁺), NAD or NADP levels, ATP, anaerobic threshold, reduced coenzyme Q, oxidized coenzyme Q; total coenzyme Q, oxidized cytochrome C, reduced cytochrome C, oxidized cytochrome C/reduced cytochrome C ratio, acetoacetate, β-hydroxy butyrate, acetoacetate/β-hydroxy butyrate ratio, 8-hydroxy-2′-deoxyguanosine (8-OHdG), levels of reactive oxygen species, levels of oxygen consumption (VO2), levels of carbon dioxide output (VCO2), and respiratory quotient (VCO2/VO2).

In certain embodiments, the level of mitochondrial activity marker is measured and the amount or frequency of administration of cysteamine product administered to a subject can be adjusted according to the level of the activity marker measured. In some embodiments, the level of mitochondrial marker is “below a target level” or “above a target level.” A target level of a mitochondrial marker is a level or range of levels of the biomarker at which a therapeutic effect is observed in the subject receiving the cysteamine product. In certain embodiments, the target level of an activity marker for a subject having an inherited mitochondrial disease or disorder is the level or range of levels of the activity marker observed in a normal, non-affected subject. In other embodiments, to indicate a therapeutic effect, the target level of a marker need not be equivalent to the level or range of levels of the marker observed in a normal subject, but can be within, e.g., 100%, 90%, 80%, 70%, 60%, 50%, 40%, 30%, 20%, 10% or 5% of the “normal” level or range of levels of the marker observed in a non-affected subject.

“Pharmaceutical composition” refers to a composition suitable for pharmaceutical use in subject animal, including humans and mammals. A pharmaceutical composition comprises a therapeutically effective amount of a cysteamine product, optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines.

As used herein “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender. In various embodiments the subject is human. In various embodiments, the subject is a child or adolescent.

### Mitochondrial Disease

Inherited mitochondrial diseases or disorders are typically associated with mutations in nuclear or mitochondrial DNA that impair respiratory chain function. Certain underlying biochemical defects of inherited or acquired mitochondrial disorders include the following signs and symptoms: increased lactate or ketone body formation, impaired ATP production, decreased respiration, increased oxidative stress and sensitivity to increased energy demand. This partial list of common elements is observed across a variety of inherited mitochondrial diseases, independent of age, gender, severity and organ system.

Exemplary inherited or acquired mitochondrial diseases include, but are not limited to, Friedreich's Ataxia, Leber's hereditary optic neuropathy, myoclonic epilepsy and ragged-red fibers, Mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes and MEGDEL syndrome (3-methylglutaconic aciduria type IV with sensorineural deafness, encephalopathy and Leigh-like syndrome). Inherited or acquired mitochondrial diseases contemplated herein exclude diseases caused by CAG repeat expansion in protein-coding portions of non-mitochondrial genes (e.g. Huntington's disease) as well as diseases that may include somatic mutations of mitochondrial DNA due to aging (e.g., Parkinson's disease, Alzheimer's disease).

Friedreich's ataxia (FRDA) is an autosomal recessive neurodegenerative disorder predominantly caused by a homozygous GAA repeat expansion mutation within intron 1 of the FXN gene (Campuzano et al., Science. 271:1423-7, 1996; Sandi et al., Neurobiol Dis. 42:496-505, 2011). Normal individuals have 5-30 GAA repeat sequences, whereas affected individuals have from approximately 70 to more than 1000 GAA triplets. The effect of the GAA expansion mutation is to reduce the production of frataxin (Campuzano et al., Hum Mol Genet. 6:1771-80, 1997), a ubiquitously expressed mitochondrial protein that is important in assembly of iron-sulfur cluster and in heme biosynthesis (Pandolfo and Pastore, J Neurol. 256 Suppl 1:9-17, 2009). Friedreich's Ataxia is viewed as representative of many inherited mitochondrial diseases, in that it reflects a broad pathology common to inherited mitochondrial diseases including multi-organ system involvement, exercise intolerance with elevated lactate, enhanced oxidative stress and biochemical lesions spanning multiple respiratory chain complexes. The disease results in progressive spinocerebellar neurodegeneration, causing symptoms of incoordination (“ataxia”), muscle weakness and sensory loss. There is also pathology of non-neuronal tissues, with cardiomyopathy a common secondary effect and diabetes found in 10% of FRDA patients (Schulz et al., Nat Rev Neurol. 5(4):222-34, 2009). Estimates of the prevalence of FRDA in the United States range from 1 in every 22,000-29,000 people to 1 in 50,000 people. Symptoms typically begin in childhood, and the disease progressively worsens as the patient grows older; patients eventually become wheelchair-bound due to motor disabilities (U.S. Pat. No. 7,968,746).

Leber's hereditary optic neuropathy (LHON) is a maternally inherited disorder with point mutations in mitochondrial DNA primarily resulting in retinal ganglion degeneration and subsequent blindness. LHON is usually due to pathogenic mitochondrial DNA (mtDNA) point mutations in the ND4, ND4L, ND1 and ND6 subunit genes of complex I of the oxidative phosphorylation chain in mitochondria. Onset of LHON typically occurs between 27 and 34 years of age and affects males more than females. Other symptoms such as cardiac abnormalities and neurological complications are also observed in some LHON patients.

Mitochondrial encephalomyopathy, lactic acidosis, and stroke-like syndrome (MELAS) is a condition that affects many of the body's systems, particularly the brain and nervous system and muscles. In most cases, the signs and symptoms of this disorder appear in childhood following a period of normal development. MELAS can result from mutations in the MT-ND1 and MT-ND5 genes, which are part of the large NADH dehydrogenase complex (complex I) in mitochondria that helps convert oxygen and simple sugars to energy. Early symptoms may include muscle weakness and pain, recurrent headaches, loss of appetite, vomiting, and seizures. Most affected individuals experience stroke-like episodes beginning before age 40. These episodes often involve temporary muscle weakness on one side of the body (hemiparesis), altered consciousness, vision abnormalities, seizures, and severe headaches resembling migraines. Repeated stroke-like episodes can progressively damage the brain, leading to vision loss, problems with movement, and a loss of intellectual function (dementia). Individuals with MELAS have a buildup of lactic acid in their bodies (lactic acidosis) and increased acidity in the blood can lead to vomiting, abdominal pain, fatigue, muscle weakness, loss of bowel control, and difficulty breathing. Less commonly, MELAS patients experience involuntary muscle spasms (myoclonus), impaired muscle coordination (ataxia), hearing loss, heart and kidney problems, diabetes, epilepsy, and hormonal imbalances.

Kearns-Sayre Syndrome (KSS) is characterized by features including typical onset before age 20, chronic, progressive, external opthalmoplegia, and pigmentary degeneration of the retina. In addition, KSS may include cardiac conduction defects, cerebellar ataxia, and raised cerebrospinal fluid (CSF) protein levels (e.g., >100 mg/dL). Additional features associated with KSS may include myopathy, dystonia, endocrine abnormalities (e.g., diabetes, growth retardation or short stature, and hypoparathyroidism), bilateral sensorineural deafness, dementia, cataracts, and proximal renal tubular acidosis.

Leigh's disease, or Leigh's Syndrome (LS), also known as Subacute Necrotizing Encephalomyelopathy (SNEM), is a rare neurometabolic disorder that affects the central nervous system. Mutations in mitochondrial DNA (mtDNA) or in nuclear DNA (SURF1[2] and some COX assembly factors) cause degradation of motor skills and eventually death. The disease usually affects infants between the age of three months and two years, and, in rare cases, teenagers and adults. The disease is characterized by dystonia (movement disorder) as well as lactic acidosis. X-linked Leigh's syndrome is caused by a mutation of the gene encoding PDHA1, part of the pyruvate dehydrogenase complex, located on the X chromosome. Recent studies have shown that certain LS patients exhibit a change in glutathione forms, including a decrease of total and reduced glutathione (GSH) and a concurrent increase in oxidized glutathione forms (GSSG+GS-Pro; OX). The patients also exhibited a decrease glutathione peroxidase activity (Genet Metab. 109(2):208-14, 2013). In some embodiments, the Leigh's syndrome patient has a POLG mutation. The disclosure contemplates treating a population of patients having a POLG mutation.

While certain mitochondrial diseases have been characterized, many diseases have had little research into the ultimate cause of the disease. Koopman et al. (EMBO J. 32(1): 9-29, 2013) describes mitochondrial and nuclear genes involved in the mitochondrial complexes and OXPHOS system as well as mutations associated with deficiencies in mitochondrial activity. It is contemplated that treatment of a subject having a mutations described in Koopman (see, e.g., Supplementary Table 1) or elsewhere in the art is treated with a cysteamine or cystamine product as described herein.

Additionally, the symptoms and manifestation of mitochondrial disease is different for different mutations in the mtDNA (Salmi et al., Scad J Clin Lab Invest, 72(2):152-7, 2012), and it has been postulated that oxidative stress contributes to pathogenesis and progression of mitochondrial diseases. Glutathione and other thiols contribute to scavenging of free radicals formed after ATP synthesis. The levels of thiols was recently investigated in children diagnosed with mitochondrial disease (Salmi et al., supra). Salmi et al. (supra) demonstrated that children with diagnosed mitochondrial disease exhibited decreased reduced/oxidized cysteine ratios, as well as reduced levels of reduced glutathione and total glutathione. Salmi, however, notes that not all mitochondrial disease patients exhibit altered thiol levels as shown in their study. Mancuso et al., (J Neurol 257:774-781, 2012) administered a whey based oral supplement (WBOS) comprising glutamylcysteine to patients diagnosed with mitochondrial disease and described that administration of WBOS decreased advanced oxidation protein products (AOPP) increased ferric reducing antioxidant power (FRAP), and increased glutathione levels. The WBOS treatment did not modify lactate levels, clinical outcome or quality of life.

Methods of treating mitochondrial disorders using Coenzyme Q10 or analogs thereof are disclosed in US Patent Publications 2011/0046219, and are currently undergoing clinical trials (Enns et al., Mol Genet Metab. 105:91-102, 2012).

In various embodiments, the effects of cysteamine products on the symptoms of inherited or acquired mitochondrial diseases or disorders are measured as improvements in disease symptoms described above. Improvement also includes slowed progression of disease symptoms. Measurement of improvement in symptoms of mitochondrial disease is carried out using routine techniques in the art, including, but not limited to, measurement of mitochondrial activity markers described below (e.g., ATP), muscle activity assays, neurological activity assays, vision assessment, cardiac activity assays (e.g., ECG), cardiac enzyme measurement, exercise tests, kidney function, blood sugar levels, blood lactate levels, and other techniques known to one of skill in the art.

Improvement in mitochondrial disease is also measured using the Newcastle Pediatric Mitochondrial Disease Scale (NPMDS) (Phoenix et al., Neuromuscul Disord. 16:814-20, 2006) which includes the following, on a scale of 0 (none) to 3 (severe): vision, hearing, feeding, motility, language, neuropathy, endocrine, gastrointestinal, encephalopathy, liver, renal, cardiovascular and respiratory function, blood enzyme levels and red blood cells, and quality of life assessment. See also Enns et al., Mol Gen Metab, 105(1):91-102, 2012.

Improvements in dystonia of patients is also measured. Dystonia is a movement disorder commonly seen in individuals with development disabilities. There are a variety of treatments available for movement disorders, but responses can differ based on the patient's cause(s) of increased muscle tone. Quantitative measures such as the Barry Albright Dystonia (BAD) scale (Barry et al., Developmental Medicine & Child Neurology 41(6):404-411, 1999) can aid in assessing and treating people with dystonia.

Neurological exams to determine neuromuscular function, which is typically compromised in patients with inherited mitochondrial diseases, are also used to assess the efficacy of cysteamine product. Standard clinical neurological/neuromuscular assessment scales will be use, such as Brain HMPAO SPECT studies.

### Cysteamine/Cystamine

Cysteamine plays a role in formation of the protein glutathione (GSH) precursor. In cystinosis, cysteamine acts by converting cystine to cysteine and cysteine-cysteamine mixed disulfide which are then both able to leave the lysosome through the cysteine and lysine transporters respectively (Gahl et al., N Engl J Med 347(2):111-21, 2002). Within the cytosol the mixed disulfide can be reduced by its reaction with glutathione and the cysteine released can be used for further GSH synthesis. The synthesis of GSH from cysteine is catalyzed by two enzymes, gamma-glutamylcysteine synthetase and GSH synthetase. This pathway occurs in almost all cell types, with the liver being the major producer and exporter of GSH. The reduced cysteine-cysteamine mixed disulfide will also release cysteamine, which, in theory is then able to re-enter the lysosome, bind more cystine and repeat the process (Dohil et al., J Pediatr 148(6):764-9, 2006). In a recent study in children with cystinosis, enteral administration of cysteamine resulted in increased plasma cysteamine levels, which subsequently caused prolonged efficacy in the lowering of leukocyte cystine levels (Dohil et al., J Pediatr 148(6):764-9, 2006). This may have been due to “re-cycling” of cysteamine when adequate amounts of drug reached the lysosome. If cysteamine acts in this fashion, then GSH production may also be significantly enhanced.

Cysteamine is a potent gastric acid-secretagogue that has been used in laboratory animals to induce duodenal ulceration; studies in humans and animals have shown that cysteamine-induced gastric acid hypersecretion is most likely mediated through hypergastrinemia. Cysteamine is currently FDA approved for use in the treatment of cystinosis, an intra-lysosomal cystine storage disorder. In previous studies performed in children with cystinosis who suffered regular upper gastrointestinal symptoms, a single oral dose of cysteamine (11-23 mg/kg) was shown to cause hypergastrinemia and a 2 to 3-fold rise in gastric acid-hypersecretion, and a 50% rise in serum gastrin levels. Symptoms suffered by these individuals included abdominal pain, heartburn, nausea, vomiting, and anorexia. U.S. Pat. No. 8,129,433 and published International Publication No. WO 2007/089670 (each of which is incorporated by reference herein in its entirety) showed that cysteamine induced hypergastrinemia arises, in part, as a local effect on the gastric antral-predominant G-cells in susceptible individuals. The data also suggest that this is also a systemic effect of gastrin release by cysteamine. Depending on the route of administration, plasma gastrin levels usually peak after intragastric delivery within 30 minutes whereas the plasma cysteamine levels peak later.

Subjects with cystinosis are required to ingest oral cysteamine (CYSTAGON®) every 6 hours day and night, or use an enteric form of cysteamine (PROCYSBI®) every 12 hours. When taken regularly, cysteamine can deplete intracellular cystine by up to 90% (as measured in circulating white blood cells), and this had been shown to reduce the rate of progression to kidney failure/transplantation and also to obviate the need for thyroid replacement therapy. Because of the difficulty in taking CYSTAGON®, reducing the required dosing improves the adherence to therapeutic regimen. International Publication No. WO 2007/089670 demonstrates that delivery of cysteamine to the small intestine reduces gastric distress and ulceration and increases AUC. Delivery of cysteamine into the small intestine is useful due to improved absorption rates from the small intestine, and/or less cysteamine undergoing hepatic first pass elimination when absorbed through the small intestine. A decrease in leukocyte cystine was observed within an hour of treatment.

In addition, sulfhydryl (SH) compounds such as cysteamine, cystamine, and glutathione are considered relevant and active intracellular antioxidants. Cysteamine protects animals against bone marrow and gastrointestinal radiation syndromes. The rationale for the importance of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. The protection is thought to result from scavenging of free radicals, either directly or via release of protein-bound GSH. An enzyme that liberates cysteamine from coenzyme A has been reported in avian liver and hog kidney. Recently, studies have reported a protective effect of cysteamine against the hepatotoxic agents acetaminophen, bromobenzene, and phalloidine.

Cystamine, in addition to its role as a radioprotectant, has been found to alleviate tremors and prolong life in mice with the gene mutation for Huntington's disease (HD). The drug may work by increasing the activity of proteins that protect nerve cells, or neurons, from degeneration. Cystamine appears to inactivate an enzyme called transglutaminase and thus results in a reduction of huntingtin protein (Nature Medicine 8, 143-149, 2002). In addition, cystamine was found to increase the levels of certain neuroprotective proteins. However, due to the current methods and formulation of delivery of cystamine, degradation and poor uptake require excessive dosing.

### Cysteamine Products

In another aspect, the disclosure provides cysteamine products for use in the methods described herein.

A “cysteamine product” in the present disclosure refers generally to cysteamine, cystamine, or a biologically active metabolite or derivative thereof, or combination of cysteamine and cystamine, and includes cysteamine or cystamine salts, esters, amides, alkylate compounds, prodrugs, analogs, phosphorylated compounds, sulfated compounds, or other chemically modified forms thereof (e.g., chemically modified forms prepared by labeling with radionucleotides or enzymes and chemically modified forms prepared by attachment of polymers such as polyethylene glycol). Thus, the cysteamine or cystamine can be administered in the form of a pharmacologically acceptable salt, ester, amide, prodrug or analog or as a combination thereof. In various embodiments, the cysteamine product includes cysteamine, cystamine or derivatives thereof. In any of the embodiments described herein, a cysteamine product may optionally exclude N-acetylcysteine.

Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula R—COOH where R is alkyl, and typically is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

### Pharmaceutical Formulations

The disclosure provides cysteamine products useful in the treatment of inherited or acquired mitochondrial diseases or disorders. To administer cysteamine products to patients or test animals, it is preferable to formulate the cysteamine products in a composition comprising one or more pharmaceutically acceptable carriers. Pharmaceutically or pharmacologically acceptable carriers or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The cysteamine products may be administered orally, parenterally, transocularly, intranasally, transdermally, transmucosally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are sterile.

Pharmaceutical compositions of the disclosure containing a cysteamine product as an active ingredient may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the disclosure.

Formulation of the pharmaceutical composition will vary according to the route of administration selected (e.g., solution, emulsion). An appropriate composition comprising the cysteamine product to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

In some embodiments, the cysteamine product disclosed herein can be lyophilized for storage and reconstituted in a suitable carrier prior to use. Any suitable lyophilization and reconstitution techniques can be employed. It is appreciated by those skilled in the art that lyophilization and reconstitution can lead to varying degrees of activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

In one embodiment, the disclosure provides use of an enterically coated cysteamine product composition. Enteric coatings prolong release until the cysteamine product reaches the intestinal tract, typically the small intestine. Because of the enteric coatings, delivery to the small intestine is improved thereby improving uptake of the active ingredient while reducing gastric side effects. Exemplary enterically coated cysteamine products are described in International Publication No. WO 2007/089670 and in International Patent Applications PCT/US14/42607 and PCT/US14/42616.

In some embodiments, the coating material is selected such that the therapeutically active agent is released when the dosage form reaches the small intestine or a region in which the pH is greater than pH 4.5. The coating may be a pH-sensitive materials, which remain intact in the lower pH environs of the stomach, but which disintegrate or dissolve at the pH commonly found in the small intestine of the patient. For example, the enteric coating material begins to dissolve in an aqueous solution at pH between about 4.5 to about 5.5. For example, pH-sensitive materials will not undergo significant dissolution until the dosage form has emptied from the stomach. The pH of the small intestine gradually increases from about 4.5 to about 6.5 in the duodenal bulb to about 7.2 in the distal portions of the small intestine. In order to provide predictable dissolution corresponding to the small intestine transit time of about 3 hours (e.g., 2-3 hours) and permit reproducible release therein, the coating should begin to dissolve at the pH range within the small intestine. Therefore, the amount of enteric polymer coating should be sufficient to substantially dissolved during the approximate three hour transit time within the small intestine, such as the proximal and mid-intestine.

Enteric coatings have been used to arrest the release of the drug from orally ingestible dosage forms. Depending upon the composition and/or thickness, the enteric coatings are resistant to stomach acid for required periods of time before they begin to disintegrate and permit release of the drug in the lower stomach or upper part of the small intestines. Examples of some enteric coatings are disclosed in U.S. Pat. No. 5,225,202 which is incorporated by reference fully herein. As set forth in U.S. Pat. No. 5,225,202, some examples of coating previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthlates, e.g., those having a free carboxyl content. See, Remington's at page 1590, and Zeitova et al. (U.S. Pat. No. 4,432,966), for descriptions of suitable enteric coating compositions. Accordingly, increased adsorption in the small intestine due to enteric coatings of cysteamine product compositions can result in improved efficacy.

Generally, the enteric coating comprises a polymeric material that prevents cysteamine product release in the low pH environment of the stomach but that ionizes at a slightly higher pH, typically a pH of 4 or 5, and thus dissolves sufficiently in the small intestines to gradually release the active agent therein. Accordingly, among the most effective enteric coating materials are polyacids having a pKa in the range of about 3 to 5. Suitable enteric coating materials include, but are not limited to, polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit RL, Eudragit RS). In one embodiment, the cysteamine product composition is administered in an oral delivery vehicle, including but not limited to, tablet or capsule form. Tablets are manufactured by first enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

The preparation of delayed, controlled or sustained/extended release forms of pharmaceutical compositions with the desired pharmacokinetic characteristics is known in the art and can be accomplished by a variety of methods. For example, oral controlled delivery systems include dissolution-controlled release (e.g., encapsulation dissolution control or matrix dissolution control), diffusion-controlled release (reservoir devices or matrix devices), ion exchange resins, osmotic controlled release or gastroretentive systems. Dissolution controlled release can be obtained, e.g., by slowing the dissolution rate of a drug in the gastrointestinal tract, incorporating the drug in an in soluble polymer, and coating drug particles or granules with polymeric materials of varying thickness. Diffusion controlled release can be obtained, e.g., by controlling diffusion through a polymeric membrane or a polymeric matrix. Osmotically controlled release can be obtained, e.g., by controlling solvent influx across a semipermeable membrane, which in turn carries the drug outside through a laser-drilled orifice. The osmotic and hydrostatic pressure differences on either side of the membrane govern fluid transport. Prolonged gastric retention may be achieved by, e.g., altering density of the formulations, bioadhesion to the stomach lining, or increasing floating time in the stomach. For further detail, see the Handbook of Pharmaceutical Controlled Release Technology, Wise, ed., Marcel Dekker, Inc., New York, N.Y. (2000), incorporated by reference herein in its entirety, e.g. Chapter 22 (“An Overview of Controlled Release Systems”).

The concentration of cysteamine product in these formulations can vary widely, for example from less than about 0.5%, usually at or at least about 1% to as much as 15 or 20% by weight and are selected primarily based on fluid volumes, manufacturing characteristics, viscosities, etc., in accordance with the particular mode of administration selected. Actual methods for preparing administrable compositions are known or apparent to those skilled in the art and are described in more detail in, for example, Remington's Pharmaceutical Science, 15th ed., Mack Publishing Company, Easton, Pa. (1980).

Compositions useful for administration may be formulated with uptake or absorption enhancers to increase their efficacy. Such enhancers include, for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

The enterically coated cysteamine product can comprise various excipients, as is well known in the pharmaceutical art, provided such excipients do not exhibit a destabilizing effect on any components in the composition. Thus, excipients such as binders, bulking agents, diluents, disintegrants, lubricants, fillers, carriers, and the like can be combined with the cysteamine product. Oral delivery vehicles contemplated for use herein include tablets, capsules, comprising the product. For solid compositions, diluents are typically necessary to increase the bulk of a tablet or capsule so that a practical size is provided for compression. Suitable diluents include dicalcium phosphate, calcium sulfate, lactose, cellulose, kaolin, mannitol, sodium chloride, dry starch and powdered sugar. Binders are used to impart cohesive qualities to a oral delivery vehicle formulation, and thus ensure that a tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, hydroxyethyl cellulose, hypromellose, and the like), and Veegum. Lubricants are used to facilitate oral delivery vehicle manufacture; examples of suitable lubricants include, for example, magnesium stearate, calcium stearate, and stearic acid, and are typically present at no more than approximately 1 weight percent relative to tablet weight. Disintegrants are used to facilitate oral delivery vehicle, (e.g., a tablet) disintegration or “breakup” after administration, and are generally starches, clays, celluloses, algins, gums or crosslinked polymers. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. If desired, flavoring, coloring and/or sweetening agents may be added as well. Other optional components for incorporation into an oral formulation herein include, but are not limited to, preservatives, suspending agents, thickening agents, and the like. Fillers include, for example, insoluble materials such as silicon dioxide, titanium oxide, alumina, talc, kaolin, powdered cellulose, microcrystalline cellulose, and the like, as well as soluble materials such as mannitol, urea, sucrose, lactose, dextrose, sodium chloride, sorbitol, and the like.

A pharmaceutical composition may also comprise a stabilizing agent such as hydroxypropyl methylcellulose or polyvinylpyrrolidone, as disclosed in U.S. Pat. No. 4,301,146. Other stabilizing agents include, but are not limited to, cellulosic polymers such as hydroxypropyl cellulose, hydroxyethyl cellulose, methyl cellulose, ethyl cellulose, cellulose acetate, cellulose acetate phthalate, cellulose acetate trimellitate, hydroxypropyl methylcellulose phthalate, microcrystalline cellulose and carboxymethylcellulose sodium; and vinyl polymers and copolymers such as polyvinyl acetate, polyvinylacetate phthalate, vinylacetate crotonic acid copolymer, and ethylene-vinyl acetate copolymers. The stabilizing agent is present in an amount effective to provide the desired stabilizing effect; generally, this means that the ratio of cysteamine product to the stabilizing agent is at least about 1:500 w/w, more commonly about 1:99 w/w.

In various embodiments, the tablet, capsule, or other oral delivery system is manufactured by enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

In various embodiments, the enterically coated cysteamine product is granulated and the granulation is compressed into a tablet or filled into a capsule. Capsule materials may be either hard or soft, and are typically sealed, such as with gelatin bands or the like. Tablets and capsules for oral use will generally include one or more commonly used excipients as discussed herein.

In a further embodiment, the cysteamine product is formulated as a capsule. In one embodiment, the capsule comprises the cysteamine product and the capsule is then enterically coated. Capsule formulations are prepared using techniques known in the art.

A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary cysteamine or cystamine product formulations contemplated for use in the present methods are described in International Patent Applications PCT/US14/42607 and PCT/US14/42616.

For administration of the dosage form, i.e., the tablet or capsule comprising the enterically coated cysteamine product, a total weight in the range of approximately 100 mg to 1000 mg is used. The dosage form is orally administered to a patient suffering from an inherited or acquired mitochondrial disorder, including, but not limited to, Friedreich's ataxia, Leber's hereditary optic neuropathy, myoclonic epilepsy and ragged-red fibers, Mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes. Inherited or acquired mitochondrial diseases contemplated herein exclude diseases caused by CAG repeat expansion in protein-coding portions of non-mitochondrial genes (e.g. Huntington's disease) as well as diseases that may include somatic mutations of mitochondrial DNA due to aging (e.g., Parkinson's disease, Alzheimer's disease).

In addition, various prodrugs can be “activated” by use of the enterically coated cysteamine. Prodrugs are pharmacologically inert, they themselves do not work in the body, but once they have been absorbed, the prodrug decomposes. The prodrug approach has been used successfully in a number of therapeutic areas including antibiotics, antihistamines and ulcer treatments. The advantage of using prodrugs is that the active agent is chemically camouflaged and no active agent is released until the drug has passed out of the gut and into the cells of the body. For example, a number of prodrugs use S—S bonds. Weak reducing agents, such as cysteamine, reduce these bonds and release the drug. Accordingly, the compositions of the disclosure are useful in combination with pro-drugs for timed release of the drug. In this aspect, a pro-drug can be administered followed by administration of an enterically coated cysteamine compositions of the disclosure (at a desired time) to activate the pro-drug.

Prodrugs of cysteamine have been described previously. See, e.g., Andersen et al., In Vitro Evaluation of Novel Cysteamine Prodrugs Targeted to g-Glutamyl Transpeptidase (poster presentation), which describes S-pivaloyl cysteamine derivatives, S-benzoyl cysteamine derivatives, S-acetyl cysteamine derivatives and S-benzoyl cysteamine)glutamate-ethyl ester). Omran et al., Bioorg Med Chem Lett. 2011 Apr. 15; 21(8):2502-4 describes a folate pro-drug of cystamine as a treatment for nephropathic cystinosis.

Thiazolidine prodrugs are also contemplated, and can be made as described previously. See e.g., Wilmore et al., J. Med. Chem., 44 (16):2661-2666, 2001 and Cardwell, W A, “Synthesis And Evaluation Of Novel Cysteamine Prodrugs” 2006, Thesis, Univ. of Sunderland.

### Dosing and Administration

The cysteamine product is administered in a therapeutically effective amount; typically, the composition is in unit dosage form. The amount of cysteamine product administered is, of course, dependent on the age, weight, and general condition of the patient, the severity of the condition being treated, and the judgment of the prescribing-physician. Suitable therapeutic amounts will be known to those skilled in the art and/or are described in the pertinent reference texts and literature. Current non-enterically coated doses are about 1.35 g/m² body surface area and are administered 4-5 times per day (Levtchenko et al., Pediatr Nephrol. 21:110-113, 2006). In one aspect, the dose is administered either one time per day or multiple times per day. The cysteamine product may be administered less than four time per day, e.g., one, two or three times per day. In some embodiments, an effective dosage of cysteamine product may be within the range of 0.01 mg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values. In some embodiments, the dose above may be the total daily dose, or may be the dose administered in one of the one, two or three daily administrations. In some embodiments, the cysteamine product is administered at a total daily dose of from approximately 0.25 g/m² to 4.0 g/m² body surface area, e.g., at least about 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, or 3.5 g/m² or may range between any two of the foregoing values. In some embodiments, the cysteamine product may be administered at a total daily dose of about 0.5-2.0 g/m² body surface area, or 1-1.5 g/m² body surface area, or 0.5-1 g/m² body surface area, or about 0.7-0.8 g/m² body surface area, or about 1.3 g/m² body surface area, or about 1.3 to about 1.95 grams/m²/day, or about 0.5 to about 1.5 grams/m²/day, or about 0.5 to about 1.0 grams/m²/day, preferably at a frequency of fewer than four times per day, e.g. three, two or one times per day. Salts or esters of the same active ingredient may vary in molecular weight depending on the type and weight of the salt or ester moiety. For administration of enteric dosage form, e.g., a tablet or capsule or other oral dosage form comprising the enterically coated cysteamine product, a total weight in the range of approximately 100 mg to 1000 mg is used. In certain embodiments, the amount of cysteamine or cystamine active ingredient in a tablet or capsule is approximately 15, 20, 25, 50, 75, 100, 125, 150, 175, 200, 250, 300, 400 or 500 mg.

The disclosure provides methods to treat inherited or acquired mitochondrial disorders in which the dosage form is administered to a patient suffering from an inherited or acquired mitochondrial disease, including, but not limited to, Friedreich's ataxia, Leber's hereditary optic neuropathy, myoclonic epilepsy and ragged-red fibers, Mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes. Inherited or acquired mitochondrial diseases contemplated herein exclude diseases caused by CAG repeat expansion in protein-coding portions of non-mitochondrial genes (e.g., Huntington's disease) as well as diseases that may include somatic mutations of mitochondrial DNA due to aging (e.g., Parkinson's disease, Alzheimer's disease). Administration may continue for at least 3 months, 6 months, 9 months, 1 year, 2 years, or more.

In some embodiments, the compositions of the disclosure are used in combination with a second drug or other therapies useful for treating mitochondrial disorders. Exemplary agents useful for the treatment of mitochondrial diseases include, but are not limited to coenzyme Q10 (CoQ10, Q10, ubiquinone), coenzyme Q10 analogs, idebenone, decylubiquinone, Epi-743, resveratrol and analogs thereof, arginine, vitamin E, tocopherol, MitoQ, glutathione peroxidase mimetics, levo-carnitine, acetyl-L-carnitine, dichloroacetate, dimethylglycine, lipoic acid, and other agents useful to treat mitochondrial diseases.

In various embodiments, the cysteamine product is administered with a second agent useful to treat underlying symptoms of a mitochondrial disease. For example, if the subject has cardiac involvement, the cysteamine product is administered with a cardiac therapeutic, including but not limited to, beta-adrenergic receptor antagonists, calcium channel blockers, ACE inhibitors or angiotensin receptor blockers.

The cysteamine product and other drugs/therapies can be administered in combination either simultaneously in a single composition or in separate compositions. Alternatively, the administration is sequential. Simultaneous administration is achieved by administering a single composition or pharmacological protein formulation that includes both the cysteamine product and other therapeutic agent(s). Alternatively, the other therapeutic agent(s) are taken separately at about the same time as a pharmacological formulation (e.g., tablet, injection or drink) of the cysteamine product.

In various alternatives, administration of the cysteamine product can precede or follow administration of the other therapeutic agent(s) by intervals ranging from minutes to hours. For example, in various embodiments, it is further contemplated that the agents are administered in a separate formulation and administered concurrently, with concurrently referring to agents given within 30 minutes of each other.

In embodiments where the other therapeutic agent(s) and the cysteamine product are administered separately, one would generally ensure that the cysteamine product and the other therapeutic agent(s) are administered within an appropriate time of one another so that both the cysteamine product and the other therapeutic agent(s) can exert, synergistically or additively, a beneficial effect on the patient. For example, in various embodiments the cysteamine product is administered within about 0.5-6 hours (before or after) of the other therapeutic agent(s). In various embodiments, the cysteamine product is administered within about 1 hour (before or after) of the other therapeutic agent(s).

In another aspect, the second agent is administered prior to administration of the cysteamine composition. Prior administration refers to administration of the second agent within the range of one week prior to treatment with cysteamine, up to 30 minutes before administration of cysteamine. It is further contemplated that the second agent is administered subsequent to administration of the cysteamine composition. Subsequent administration is meant to describe administration from 30 minutes after cysteamine treatment up to one week after cysteamine administration.

It is further contemplated that other adjunct therapies may be administered, where appropriate. For example, the patient may also be administered a diabetic diet or food plan, surgical therapy, or radiation therapy where appropriate.

The effectiveness of a method or composition of the described herein can be assessed, for example, by measuring mitochondrial activity marker activity levels. Additional measures of the efficacy of the methods of the disclosure include assessing relief of symptoms associated with inherited or acquired mitochondrial diseases or disorders, including, but not limited to, Friedreich's ataxia, Leber's hereditary optic neuropathy, myoclonic epilepsy and ragged-red fibers, mitochondrial encephalomyopathy, lactic acidosis, and stroke-like syndrome (MELAS), Kearn-Sayre syndrome, subacute necrotizing encephalopathy (Leigh's Syndrome), and mitochondrial cardiomyopathies and other syndromes due to multiple mitochondrial DNA deletions. Additional mitochondrial diseases include neurogenic muscle weakness, ataxia and retinitis pigmentosa (NARP), progressive external opthalmoplegia (PEO), and Complex I disease, Complex II disease, Complex III disease, Complex IV disease and Complex V disease, which relates to dysfunction of the OXPHOS complexes and MEGDEL syndrome (3-methylglutaconic aciduria type IV with sensorineural deafness, encephalopathy and Leigh-like syndrome). Inherited or acquired mitochondrial diseases contemplated herein exclude diseases caused by CAG repeat expansion in protein-coding portions of non-mitochondrial genes (e.g. Huntington's disease) as well as diseases that may include somatic mutations of mitochondrial DNA due to aging (e.g., Parkinson's disease, Alzheimer's disease).

Hyperlactaemia (high blood lactate levels) is characterized by levels from 2 mmols/L to 5 mmols/L. Lactic acidosis is considered severe when levels are greater than 5 mmols/L; such levels are associated with an increased mortality rate.

In various embodiments, the effects of cysteamine products on the symptoms of inherited or acquired mitochondrial diseases or disorders are measured as improvements in disease symptoms described above. Assessment of improvement also includes slowed progression of disease symptoms. Measurement of mitochondrial disease symptoms is carried out using routine techniques in the art, including, but not limited to, measurement of mitochondrial activity markers described below (e.g., ATP), improvement in any muscle activity, neurological activity, vision, cardiac activity, cardiac enzymes, exercise tests, and other techniques known to one of skill in the art.

Improvement in mitochondrial disease is also measured using the Newcastle Pediatric Mitochondrial Disease Scale (NPMDS) (Phoenix et al., Neuromuscul Disord. 16:814-20, 2006) which includes the following, on a scale of 0 (none) to 3 (severe): vision, hearing, feeding, motility, language, neuropathy, endocrine, gastrointestinal, encephalopathy, liver, renal, and cardiovascular, respiratory function, blood enzymes and red blood cells, and quality of life assessment. See also Enns et al., Mol Gen Metab, 105(1):91-102, 2012.

Improvements in dystonia symptoms is also measured using the Barry Albright Dystonia (BAD) scale (Barry et al., Developmental Medicine & Child Neurology 41(6):404-411, 1999).

Neurological exams to determine neuromuscular function, which is typically compromised in patients with inherited mitochondrial diseases, are also used to assess the efficacy of cysteamine product. Standard clinical neurological/neuromuscular assessment scales will be use, such as Brain HMPAO SPECT studies (Enns et al., Mol Gen Metab, 105(1):91-102, 2012).

In various aspects, in order to assess the efficacy of the cysteamine products on mitochondrial disease, levels of mitochondrial activity markers are measured in a sample (e.g., whole blood, plasma, cerebrospinal fluid, or cerebral ventricular fluid). Mitochondrial activity markers include, but are not limited to, free thiol levels, glutathione (GSH), reduced glutathione (GSSH), total glutathione, advanced oxidation protein products (AOPP), ferric reducing antioxidant power (FRAP), lactic acid, pyruvic acid, lactate/pyruvate ratios, phosphocreatine, NADH(NADH+H⁺) or NADPH(NADPH+H⁺), NAD or NADP levels, ATP, anaerobic threshold, reduced coenzyme Q, oxidized coenzyme Q; total coenzyme Q, oxidized cytochrome C, reduced cytochrome C, oxidized cytochrome C/reduced cytochrome C ratio, acetoacetate, β-hydroxy butyrate, acetoacetate/β-hydroxy butyrate ratio (ketone body ratio), 8-hydroxy-2′-deoxyguanosine (8-OHdG), levels of reactive oxygen species, levels of oxygen consumption (VO2), levels of carbon dioxide output (VCO2), and respiratory quotient (VCO2/VO2).

Exercise intolerance is also a useful means to determine the efficacy of administration of a cysteamine product, where an improvement in exercise tolerance (i.e., a decrease in exercise intolerance) indicates efficacy of a given therapy. One of the characteristics of mitochondrial myopathies is a reduction in maximal whole body oxygen consumption (VO2ₘₐₓ) (Taivassalo et al., Brain 126:413-23, 2003), and most mitochondrial myopathies show a characteristic deficit in peripheral oxygen extraction (A-V O2 difference) and an enhanced oxygen delivery (hyperkinetic circulation). This can be demonstrated by a lack of exercise induced deoxygenation of venous blood with direct AV balance measurements (Taivassalo et al., Ann. Neurol. 51:38-44, 2002) and non-invasively by near infrared spectroscopy (Lynch et al., Muscle Nerve 25:664-73, 2002); van Beekvelt et al., Ann. Neurol. 46:667-70, 1999).

Additional assays to measure mitochondrial activity markers are disclosed in U.S. Pat. No. 7,968,746.

### Animal Models

Cysteamine products can be evaluated in animal models known in the art for the disease indications contemplated herein.

For example, Marella et al. (PLoS One. 5:e11472, 2010) disclose a rat model for Leber's optic neuropathy. Dyer et al., (Brain Res Mol Brain Res. 132:208-20, 2004) disclose a model of Leber congenital amaurosis (LCA) having a mutation in the aryl-hydrocarbon interacting protein-like 1 (AIPL1) gene, which is also seen in human disease.

Seznec et al., (Hum Mol Genet. 13:1017-24, 2004) have developed frataxin (FXN) deficient mice that develop iron accumulation and isolated cardiac disease, similar to symptoms observed in FRDA patients. Sandi et al., (Neurobiol. Dis. 42:496-505, 2011) have investigated the effects of histone deacetylase (HDAC) inhibitors in mouse model having a GAA repeat expansion mutation. Mice (YG8R) are generated by cross breeding YG8 human genomic YAC transgenic mice that contain the entire FXN gene and expanded GAA repeats with heterozygous Fxn knockout mice (Cossee et al., Hum Mol Genet. 9:1219-26, 2000). The resulting YG8R mice rescue the embryonic lethality of the Fxn homozygous knockout alleles by expressing only human frataxin from the GAA repeat-mutated FXN transgene in a mouse frataxin null background.

A model for Leigh Syndrome is disclosed in Johnson et al. (Science 342(6165):1524-28, 2013) using mice that display a mutation the Ndufs4 gene (Ndufs4−/−) mouse. Ndufs4 encodes a protein involved in the activity of complex I of the mitochondrial electron transport chain. Ndufs4−/− mice exhibit a progressive neurodegenerative phenotype characterized by lethargy, ataxia and weight loss, eventually leading to death. See also Quintana et al., Proc. Natl. Acad. Sci. U.S.A. 107:10996-11001, 2010.

### Kits

The disclosure also provides kits for carrying out the methods of the disclosure. In various embodiments, the kit contains, e.g., bottles, vials, ampoules, tubes, cartridges and/or syringes that comprise a liquid (e.g., sterile injectable) formulation or a solid (e.g., tablet, capsule, lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a cysteamine product-containing composition. The kits can also include dispensing devices, such as aerosol or injection dispensing devices, pen injectors, autoinjectors, needleless injectors, syringes, and/or needles. In various embodiments, the kit also provides an oral dosage form, e.g., a tablet or capsule or other oral formulation described herein, of the cysteamine product for use in the method. The kit also provides instructions for use.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

### EXAMPLES

### Example 1

### Yeast-Based Screen for Effects of Cysteamine on Mitochondria Activity

Representative animal models of many inherited mitochondrial disease are not available for testing the efficacy of a candidate drug molecule. As such, yeast-based assays have been used to determine the effects of molecules on mitochondria activity due to the conservation of the activity and genome of the human mitochondria and yeast mitochondria (Couplan et al., Proc Natl Acad Sci USA 108:11989-94, 2011).

For example, a yeast model of ATP synthase disruption, resembling that in NARP (neuropathy, ataxia and retinitis pigmentosa) is disclosed in Couplan et al., (Proc Natl Acad Sci USA, supra). Additionally, frataxin-knockout yeast (Marobbio et al., Mitochondrion 12(1):156-61, 2012) exhibit mitochondrial iron accumulation, iron-sulfur cluster defects and high sensitivity to oxidative stress similar to frataxin-deficient human mitochondria, and are useful to determine the efficacy of compounds on inhibiting the effects of frataxin deficiency on the cell.

Using the above assays as well as other strains of yeast undergoing oxidative stress that is similar to that in human mitochondria, the effects of administration of a cysteamine product are assessed. It is expected that cysteamine will alleviate one or more symptoms of the oxidative stress in the cell and increase cell growth and viability.

### Example 2

### Effects of Cysteamine in Leber Hereditary Optic Neuropathy (LHON)

Leber Hereditary Optic Neuropathy (LHON) results from one of several mutations in mitochondrial DNA that leads to disruption of the mitochondrial respiratory chain and damage to the retinal ganglion cells (Sadun et al., Arch Neurol 69:331-38, 2012).

In order to determine the effects of cysteamine compositions on the progression of LHON and loss of vision in patients, LHON-affected individuals are administered a cysteamine composition and clinical symptoms monitored as described in Sadun et al. (supra).

Briefly, patients are administered a cysteamine composition orally, or topically using cysteamine eyedrops (Tavares et al., Cornea 28:938-40, 2009), at an appropriate dosage, e.g., 25, 50, 100, 200, 250, or 300 mg/dose, and may be administered the cysteamine composition 1, 2 or 3 or more times a day as necessary. Administration of the cysteamine composition is continued for at least 1, 2, 3, 4, 5, or 6 months or 1 year or more. During treatment, patients are monitored for improvement of or slowed decrease in visual acuity and visual field (Sadun, supra) compared to those without treatment.

It is contemplated that administration of the cysteamine composition will improve visual acuity and slow the progression of retinal dysfunction in LHON patients.

### Example 3

### Effects of Cysteamine on Friedrich's Ataxia

Fibroblasts from Friedrich's Ataxia (FRDA) patients have been shown to be sensitive to inhibition of the de novo synthesis of glutathione (GSH) with L-buthionine-(S,R)-sulfoximine (BSO), a specific inhibitor of GSH synthetase (Jauslin et al., Hum. Mol. Genet. 11(24):3055-3063, 2002). Contact of FRDA fibroblasts with BSO leads to conditions mimicking oxidative stress and induces cell death due to inhibited cell respiration. It has been shown that preincubation of FRDA fibroblasts with idebenone (a CoQ10 analog) or vitamin E prior to exposure to BSO protected cells from cell death. However, not all antioxidants tested induced the same level of protection from oxidative stress (Jauslin et al., supra).

To measure the effects of cysteamine products on FRDA cells, a cysteamine product is administered to cultured FRDA fibroblasts after sensitization with BSO and the resulting glutathione synthesis and cell viability measured. An increase in cell viability indicates that cysteamine is able to rescue the oxidative stress in FRDA cells and is serves as a potential therapeutic to treat FRDA patients.

The effects of cysteamine on Friedrich'a ataxia is also assessed using frataxin deficient animal models (Seznec et al., Hum Mol Genet. 13:1017-24, 2004). Frataxin deficient mice develop iron accumulation after onset of pathology and isolated cardiac disease, similar to symptoms observed in FRDA patients. The effects of cysteamine administration on iron accumulation, cardiac pathology and mitochondrial activity markers in frataxin deficient animals is measured using techniques known in the art (Seznec et al., supra), and an improvement in FDRA symptoms indicates cysteamine and related compounds are useful to treat FDRA and other mitochondrial diseases.

### Example 4

### Administration of Cysteamine to Superoxide Dismutase Null (SOD2) Mice

In order to assess the effects of cysteamine on the mitochondrial oxidation pathway, cysteamine bitartrate is administered to mice having mutations in the superoxide dismutase gene (Sod2 null mice) and survival, weight gain, and toxicity measured.

Sod2 null mice provide a method for determining the efficacy in vivo of compounds having antioxidant properties, particularly those with mitochondrial efficacy. Without antioxidant efficacy, Sod2 null mice die after approximately 1 week, with antioxidant intervention, the lifespan can be extended 3-fold using powerful catalytic synthetic antioxidants such as EUK-189 (Melov et al., J Neurosci. 21(21):8348-53, 2001).

The following groups of mice are treated: Group 1: Cysteamine Bitartrate (30 mg/kg) treated Sod2 null mice; Group 2: Vehicle treated Sod2 wild-type mice; Group 3: Cysteamine Bitartrate treated Sod2 heterozygotes, and wild-type controls. A single dose of the test agent is administered to the animals, either intraperitoneally or subcutaneously.

In an initial experiment, administration of cysteamine did not result in toxicity or abnormalities, and weight gain was normal compared to untreated animals. Survival analysis of the preliminary experiment was inconclusive.

Additional experiments are carried out using multiple does and altered dose regimens to determine the effects of the cysteamine product on survival in Sod2 null animals.

### Example 5

### Administration of Cysteamine to Patients with Mitochondrial Disease

Inherited mitochondrial diseases are the majority of mitochondrial diseases (or called mitochondrial cytopathies), a collection (>40) of energy metabolism disorders. They are the result of defects in mitochondrial DNA (for maternal inheritance) or nuclear DNA (for autosomal inheritance) coding for electron transport chain proteins or other molecules needed for mitochondrial function. Their clinical manifestations are extremely diverse and to various degrees of severity, and often involve multiple different tissues, particularly in cells that require high energy such as brain and muscles. Despite their distinct clinical manifestations, mitochondrial diseases share a common feature that mitochondria's ability to produce energy is damaged and consequently the mitochondria is further damaged due to subsequent byproducts accumulation and interference with other chemical reactions in the cells. They are estimated to have a prevalence of 1:5000 to 1:10,000; with approximately 1,000 to 4,000 children born with them in the United States each year. The age of onset varies from early infancy to adulthood, and typically by age of ten, approximately one in 4,000 American children is diagnosed. Available therapies remain supportive and none is effective in curing. (Salmi et al., supra)

A recent study in a cohort of children with biochemically and/or genetically confirmed mitochondrial diseases found that their plasma thiols and their redox state are altered, indicating an increase in oxidative stress and depletion of antioxidant supplies (Salmi et al., 72(2):152-157, 2012). The ability of cysteamine to increase cellular thiol pool can potentially address the relative thiol deficiency in those patients and likely to address the underlying pathophysiology of the diseases. Moreover, in a recent publication about a new compound, EPI-743, that seems to have some efficacy in Leigh syndrome, (Martinelli et al. Mol Genet Metab. 107(3):383-388, 2012) the authors concluded that data support glutathione as a “redox blood signature” in mitochondrial disorders and its use as a clinical trial endpoint in the development of mitochondrial disease therapies (Pastore et al., Mol Genet Metab. Mar. 24, 2013).

Cysteamine is an aminothiol that participates in a thiol-disulfide interchange reaction converting cystine into cysteine and cysteine-cysteamine mixed disulfide. This cysteine-cysteamine mixed disulfide can exit the lysosome through the lysosome membrane (Gahl et al., Biochem J. 228(3):545-550, 1985), as it is transported through the intestinal barrier or the blood brain barrier, by the lysine transporter (Pinto et al., J Neurochem. 94(4):1087-1101, 2005; Bousquet et al., J Neurochem. 114(6):1651-1658, 2010) or a lysine-like transporter, the PQLC2 protein (Jézégou et al., Proc Nat Acad Sci. 109(50):E3434-E3443, 2012). This mechanism is the rationale that has been successfully used to treat patients with cystinosis for more than 20 years. This biochemical reaction results in an increase of the cellular thiol pool, making more cysteine available for glutathione (GSH) synthesis (Maher et al., J Neurochem. 107(3):690-700, 2008). Glutathione is composed of the amino acids cysteine, glutamate and glycine (Maher et al., supra). The availability of cysteine, which exists primarily as cystine, is the major rate-limiting factor in GSH production (Armstrong et al., Invest Ophthalmol Vis Sci. 45(11):4183-4189, 2004). Recent findings by Mancuso et al. reinforce the notions that in mitochondrial diseases oxidative stress is important and can be reduced by administration of a cysteine donor (Mancuso et al., J Neurol. 257(5):774-781, 2010).

In order to evaluate the efficacy of cysteamine in treating inherited mitochondrial disorders, a Phase 2b clinical trial is conducted. Patients are chosen based on pre-determined inclusion/exclusion criteria.

Patients (male or female) with either a documented genetically confirmed diagnosis of inherited mitochondrial diseases OR clinical diagnosis meeting the diagnostic criteria of respiratory chain disorder “definite” on “Mitochondrial Disease Criteria” in the absence of genetic confirmation, who are >2 years old, and meet other specified inclusion and exclusion criteria, are included in this study. Diagnosis of a mitochondrial disease can be carried out according to criteria set forth in Wolf N I, Smeitink J A. (Mitochondrial disorders: a proposal for consensus diagnostic criteria in infants and children. Neurology. 59(9):1402-1405, 2002). This system allocates points based on appearance of particular symptoms, the final calculation of points results in the following diagnosis: 1 point, respiratory chain disorder unlikely; 2-4 points, respiratory chain disorder possible; 5-7 points, respiratory chain disorder probable; 8-12 points, respiratory chain disorder definite. Exemplary areas measured include, but are not limited to, muscular presentation (muscular signs and symptoms, max. 2 points; CNS presentation (max. 2 points, 1 point each); multisystemic involvement (max. 3 points, 1 point each system), such as haematology, gastrointestinal tract, heart, kidney, eyes, ears and peripheral nervous system; Metabolic and other investigations (4 points at maximum); and morphology (4 points at maximum).

Patients with inherited mitochondrial diseases associated with nuclear or mitochondrial DNA mutations that impair the respiratory chain are included. These include, but are not limited to the following clinical syndromes: Friedreich's ataxia; Leber's hereditary optic neuropathy; myoclonic epilepsy and ragged-red fibers (MERFF); mitochondrial encephalomyopathy, lactic acidosis, and stroke-like syndrome (MELAS); Kearn-Sayre syndrome; subacute necrotizing encephalopathy (Leigh's Syndrome); others, e.g., mitochondrial cardiomyopathies and other syndromes due to multiple mitochondrial DNA deletions. Up to 12 patients will be enrolled if there is no toxicity up to the level of 1300 mg/day of delayed release cysteamine.

This study is conducted in compliance with the protocol approved by the local Institutional Review Boards (IRB) or Ethics Committees (EC), and according to FDA and ICH Good Clinical Practice guidelines.

In one aspect of the study, an enteric coated cysteamine composition is administered to patients twice daily, e.g., every 12 hours, for a period of approximately 12 weeks. The study will evaluate safety and tolerability of the cysteamine therapeutic administered up to 1.3 gram/m²/day in two divided doses, every 12 hours, for up to 3 months in patients with inherited mitochondrial disease. The study will also set out to characterize the pharmacokinetics (PK) and pharmacodynamics (PD) of the cysteamine therapeutic in patients with inherited mitochondrial diseases at steady state, on a stable dose of cysteamine.

Subjects will undergo screening procedures (day −28 to day −1) to determine if they are eligible for the study, including review of inclusion/exclusion criteria, a recorded medical history, including history of inherited mitochondrial diseases and family history, calculation of BMI and body surface area, physical examination, measurement of vital signs (blood pressure, heart rate, respiratory rate, and oral body temperature), and obtaining a 12-lead ECG.

A primary outcome measure is quality of life based upon the Newcastle Paediatric Mitochondrial Disease Scale (NPMDS) for ages 2-11 years. Secondary Endpoint measurements include: neuromuscular function as evaluated with Barry Albright Dystonia Scale (Barry et al., Developmental Medicine & Child Neurol 41(6):404-411, 1999). The change in performance on these test scales are measured between day 1 and the last (6th) bi-monthly visit. Also measured bi-weekly are the level of lactate, pyruvate and lactate/pyruvate ratio; ketone body ratio; blood levels of glutathione; analysis of oxidative stress biomarkers, including advanced oxidation protein products (AOPP) and ferric reducing antioxidant power (FRAP), 10,8-hydroxy-2′-deoxyguanosine (8-OHdG), and threshold to collagen-induced aggregation of platelets (Hayes et al., The American Journal of Clinical Nutrition. 49(6):1211-1216, 1989).

Cysteamine Dose Increase Methodology:

Delayed release cysteamine will be administered following a Fibonacci dose-escalation design over 6 weeks with a progressive weekly dose increase (0.1, 0.2, 0.3, 0.5, 0.8, 1.3 g/m²/day), and then patients will stay at their highest tolerated dose for up to 3 months.

Cysteamine Dose Decrease Methodology:

Delayed release cysteamine dose decrease will be allowed if during a one week-course, the patient experiences a grade II toxicity or worse, the dose is reduced to the dose level of the previous week period.

After Day 1 screening, the patient will return to the clinical site every 2 weeks for a bi-monthly visit. At this bi-monthly visit, the following assessments will be conducted: measure height and weight, calculate BMI and body surface area, perform physical examination, measurement of vital signs (blood pressure, heart rate, respiratory rate, and oral body temperature), obtain a 12-lead ECG, and obtain blood sample for PD biomarkers (lactate, pyruvate, ketone, glutathione, AOPP, FRAP, 8-OHdG and platelets). BMI is calculated using the following formula: BMI=weight (kg)÷ height (m)². To calculate body surface area (m²) the method of Haycock can be used [Haycock G B, et al., J Pediatr. 93(1):62-6, 1978], m²=[Height (cm) 0.3964×Weight (kg) 0.5378]*0.024265.

At every other bi-monthly visit (i.e., at month 1, 2 and 3) the following are determined: clinical laboratory tests (serum chemistry, hematology, and urinalysis); administer NPMDS and Barry Albright Dystonia Scale, and record concomitant medications and monitor adverse events (AEs). Exemplary tests are set out in the following Table

Clinical Laboratory Tests

| Hematology | Serum Chemistry | Urinalysis |
|---|---|---|
| Hematocrit | Alanine | Bilirubin |
| | aminotransferase | |
| Hemoglobin | Albumin | Blood (qualitative) |
| Mean corpuscular | Aspartate | Color |
| hemoglobin | aminotransferase | |
| Mean corpuscular | Alkaline phosphatase | Glucose |
| hemoglobin concentration | | |
| Mean corpuscular volume | Amylase | Ketones |
| Erythrocytes | Conjugated bilirubin | Leukocyte esterase |
| Red cell distribution width | Total bilirubin | Nitrite |
| Platelet count | Blood urea nitrogen | pH |
| Differential (absolute, %) | Calcium | Protein |
| Leukocytes | Bicarbonate | Specific gravity |
| Basophils | Chloride | Turbidity |
| Eosinophils | Total cholesterol* | Urobilinogen |
| Lymphocytes | Creatinine | Microscopic |
| | | examination |
| Monocytes | Gamma glutamyl | |
| | transpeptidase | |
| Neutrophils | Glucose | |
| Reticulocyte count* | Lactate dehydrogenase | |
| | Phosphorus | |
| | Potassium | |
| | Total protein | |
| | Sodium | |
| | Triglycerides* | |
| | Uric acid | |

Other Laboratory Tests

| | |
|---|---|
| Serum pregnancy test | Human chorionic gonadotropin |

Exemplary assays for measuring the recited endpoints are recited below. Additional assays know in the art can also be used to measure the recited endpoint.

Blood Volume:

The estimated volume of blood drawn per sample for the subject will be approximately 4.5 mL for Initial Visit tests (i.e., clinical laboratory tests), 0.5 mL for serum pregnancy tests, 3.0 mL for safety clinical laboratory tests (i.e., clinical laboratory tests), 3.0 mL for Study Termination tests.

12-Lead Electrocardiograms:

Standard 12-lead ECGs are used for the ECG evaluation. All scheduled ECGs should be performed after the subject has rested quietly in the supine position for at least 5 minutes. A single, 10 second, 12-lead ECG is obtained on all subjects. The ECGs are recorded at the specified timepoints at a speed of 25 mm/sec and amplitude of 10 mm/mV.

Physical Examinations:

The physical examination includes assessments of the following: general appearance, eyes, ears, nose and throat, chest (heart, lungs), abdomen (palpation, GI sounds), extremities and skin. A basic neurological examination is also conducted.

Vital Signs:

Blood pressure may be measured in the seated position. Screening blood pressure may be retested 3 times at intervals of no less than 5 minutes between each measurement. Vital signs (systolic/diastolic blood pressure, heart rate, respiratory rate, and oral body temperature) are measured according standard protocols. Blood pressure is preferably measured with the arm supported at the level of the heart, and recorded to the nearest 1 mm Hg. The subject should be at rest for at least 5 minutes before the blood pressure is measured. The use of automated devices for measuring blood pressure and heart rate are acceptable. When done manually, heart rate is measured in the brachial or radial artery for at least 30 seconds.

Newcastle Pediatric Mitochondrial Disease Scale (NPMDS):

The NPMDS has been introduced to allow evaluation of the progression of mitochondrial disease in patients less than 18 years of age. (The Newcastle Mitochondrial Disease Scale (NMDS) provides a similar assessment tool for adult patients). In the pediatric population, demonstrating a genetic or biochemical basis for mitochondrial disease can be very difficult. It is recommended that the scale be administered to patients where there is a strong clinical suspicion of mitochondrial disease as well as those with a confirmed (biochemical or genetic) diagnosis. Repeated administration of the scale permits the longitudinal monitoring of these patients.

The rating scale encompasses many aspects of mitochondrial disease by exploring several domains: Current Function; System Specific Involvement; Current Clinical Assessment and Quality of Life. Almost every question in the scale has a possible score from 0-3: 0 representing normal, 1—mild, 2—moderate and 3—severe. In each case, examples of mild, moderate and severe impairment or disability are given. Three age-specific versions of the NPMDS, 0-24 months, 2-11 years and 12-18 years are used as appropriate.

Barry Albright Dystonia Scale:

Dystonia is a movement disorder commonly seen in individuals with development disabilities. There are a variety of treatments available for movement disorders, but responses can differ based on the patient's cause(s) of increased muscle tone. Quantitative measures such as the Barry Albright Dystonia (BAD) scale (Barry et al., Developmental Medicine & Child Neurology 41(6):404-411, 1999) can aid in assessing and treating people with dystonia. The BAD scale is an appropriate quantitative measurement tool to assess patient's dystonia who do not have voluntary control of their movements, and have significant cognitive impairment.

Biomarkers in mitochondrial disease can be measured as follows.

Level of Lactate, Pyruvate and Lactate/Pyruvate Ratio:

Lactic acid is produced by reduction of pyruvate, a product of anaerobic metabolism of glucose, and oxidative metabolism of pyruvate proceeds partly through the mitochondrial respiratory chain. Dysfunction of the respiratory chain may lead to inadequate removal of lactate and pyruvate from the circulation and elevated lactate/pyruvate ratios are observed in mitochondrial cytopathies (Scriver C R. The metabolic and molecular bases of inherited disease. 7th ed. New York: McGraw-Hill, Health Professions Division; 1995; Munnich et al., J Inherit Metab Dis. 15(4):448-455, 1992). Blood lactate/pyruvate ratio (Chariot et al., Arch Pathol Lab Med. 118(7):695-697, 1994) is, therefore, widely used as a noninvasive test for detection of mitochondrial cytopathies and toxic mitochondrial myopathies (Chariot et al., Arthritis Rheum. 37(4):583-586, 1994).

For pyruvate, blood must immediately be precipitated with perchloric acid, at the bedside. Blood lactate is stable in fluoride/oxalate samples for at least 3 hours at room temperature. It is much less stable when collected into heparinized tubes. In one aspect, it will be clear that blood lactate is likely to be high in children who have been physically active particularly if they were struggling during venepuncture, so every precaution will be taken to prevent struggling as much as possible.

Ketone Body Ratio:

Changes in the redox state of liver mitochondria can be investigated by measuring the arterial ketone body ratio (acetoacetate/3-hydroxybutyrate: AKBR) (Ueda et al., J Cardiol. 29(2):95-102, 1997).

8-hydroxy-2′-deoxyguanosine (8-OHdG):

Plasma and urine specimens for each patient are protected from light and stored at −80° C. Samples are analyzed for the level of 8-hydroxy-2′-deoxyguanosine (8-OHdG). 8-OHdG is formed from a hydroxyl radical attack at the C-8 position of deoxyguanosine in DNA (Kasai et al., Carcinogenesis. 7(11):1849-1851, 1986). Urinary excretion of 8-OHdG often has been used as a biomarker to assess the extent of repair of ROS-induced DNA damage in both the clinical and occupational setting (Erhola et al., FEBS Lett. 409(2):287-291, 1997; Honda et al., Leuk Res.; 24(6):461-468, 2000; Pilger et al., Free Radic Res. 35(3):273-280, 2001; Kim et al., Environ Health Perspect. 112(6):666-671, 2004).

Advanced Oxidation Protein Products (AOPP):

(Mancuso et al., J Neurol. 257(5):774-781, 2010) Advanced oxidation protein products are the result of protein oxidation by reactive oxygen species. Plasma AOPP are related to dityrosine, a marker of oxidative damage to proteins, and are present in plasma in two distinct forms, 670 and 70 kDa in molecular weight, corresponding respectively to albumin aggregates and albumin monomeric form. Increases in plasma AOPP have been reported in renal failure, and in neurodegenerative disorders involving mitochondrial dysfunction and oxidative stress, such as amyotrophic lateral sclerosis.

Ferric Reducing Antioxidant Power (FRAP):

(Mancuso et al., J Neurol. 257(5):774-781, 2010) Ferric reducing antioxidant power levels provide estimates of the total plasma antioxidant capability. The FRAP test measures the combined effect of non-enzymatic antioxidants, providing an index of the intrinsic ability to prevent oxidative damage.

Adverse events will also be measured using appropriate criteria. Adverse events include skin rash, skin lesions, seizure, lethargy, somnolence, depression, encephalopathy, gastrointestinal ulceration and/or bleeding, nausea, vomiting, loss of appetite (anorexia), diarrhea, fever, and abdominal pain. The severity of AEs is categorized using the Common Terminology Criteria for Adverse Events (CTCAE), Version 3.0 [Cancer Therapy Evaluation Program, 2003] or otherwise as follows: MILD (Grade 1): experience is minor and does not cause significant discomfort to subject or change in activities of daily living (ADL); subject is aware of symptoms but symptoms are easily tolerated; MODERATE (Grade 2): experience is an inconvenience or concern to the subject and causes interference with ADL, but the subject is able to continue with ADL; SEVERE (Grade 3): experience significantly interferes with ADL and the subject is incapacitated and/or unable to continue with ADL; LIFE THREATENING (Grade 4): experience that, in the view of the Investigator, places the subject at immediate risk of death from the event as it occurred (i.e., it does not include an event that had it occurred in a more severe form, might have caused death). By the CTCAE criteria defined above, the Grade 5 category is death

The safety profile of delayed release cysteamine is investigated by changes from the last study visit as noted in the following safety assessments: physical examination, vital signs, ECG and clinical laboratory testing.

### Example 6

### Treatment of Leigh's Syndrome Patients with Cysteamine

Leigh's syndrome is a neurometabolic disorder affecting the central nervous system and is thought to be cause by mutations in mitochondrial DNA (mtDNA) or in nuclear DNA (SURF1[2] and some COX assembly factors). These mutations cause degradation of motor skills and eventually death. The disease usually affects infants between the age of three months and two years, and, in rare cases, teenagers and adults. The disease is characterized by dystonia (movement disorder) as well as lactic acidosis. X-linked Leigh's syndrome is caused by a mutation of the gene encoding PDHA1, part of the pyruvate dehydrogenase complex, located on the X chromosome.

Patients diagnosed as having Leigh's syndrome were treated with cysteamine at previously determined tolerable doses. An 11 year old female with a POLG mutation was orally administered 600 mg delayed release cysteamine daily (8 tablets×75 mg) for nine weeks. No new adverse events or seizures were reported during the study period. The patient and family noted improvement in running and walking ability while receiving cysteamine therapy. The patient's appetite also increased while on cysteamine therapy.

A 9 year old male has also been treated daily with 450 mg delayed release cysteamine taken orally (six tablets of 75 mg) for 9 weeks. A slight regression in speech was noted shortly after therapy began, and no change in disease symptoms have been observed to date in this patient.

Additional studies measuring levels of lactate, pyruvate and lactate/pyruvate ratio; ketone body ratio; blood levels of glutathione; analysis of oxidative stress biomarkers, including advanced oxidation protein products (AOPP) and ferric reducing antioxidant power (FRAP), 10,8-hydroxy-2′-deoxyguanosine (8-OHdG), and threshold to collagen-induced aggregation of platelets are performed on the treated subjects.

The results described herein demonstrate that cysteamine therapy is useful to treat symptoms of inherited mitochondrial disease.

Numerous modifications and variations in the invention as set forth in the above illustrative examples are expected to occur to those skilled in the art. Consequently only such limitations as appear in the appended claims should be placed on the invention.

1. A method of treating an inherited or acquired mitochondrial disorder comprising, administering an effective amount of cysteamine or a derivative thereof or cystamine or a derivative thereof to a subject suffering from an inherited or acquired mitochondrial disorder.

2. The method of claim 1, wherein the inherited mitochondrial disorder is selected from the group consisting of Friedreich's ataxia, Leber's hereditary optic neuropathy (LHON), myoclonic epilepsy and ragged-red fibers, mitochondrial encephalomyopathy, lactic acidosis, and stroke-like syndrome (MELAS), Kearn-Sayre syndrome and subacute necrotizing encephalopathy (Leigh's Syndrome).

3. The method of claim 1, wherein the method comprises administering cysteamine or a derivative thereof.

4. The method of claim 1, wherein the cysteamine or derivative thereof or cystamine or derivative thereof is administered orally.

5. The method of claim 1, wherein the cysteamine or derivative thereof or cystamine or derivative thereof is a delayed release cysteamine composition.

6. The method of claim 5, wherein the delayed or controlled release dosage form comprises an enteric coating that releases the cysteamine composition when the composition reaches the small intestine or a region of the gastrointestinal tract of a subject in which the pH is greater than about pH 4.5.

7. The method of claim 1, wherein the cysteamine or derivative thereof or cystamine or derivative thereof is administered less than four times per day.

8. The method of claim 1, wherein the cysteamine or derivative thereof or cystamine or derivative thereof is administered twice a day.

9. The method of claim 1, wherein the subject has decreased thiol levels compared to a non-affected subject.

10. The method of claim 1, wherein the administering results in improvement in mitochondrial activity markers compared to levels before administration of the cysteamine or derivative thereof or cystamine or derivative thereof.

11. The method of claim 10, wherein the mitochondrial activity marker is selected from the group consisting free thiol levels, glutathione (GSH), reduced glutathione (GSSH), total glutathione, advanced oxidation protein products (AOPP), ferric reducing antioxidant power (FRAP), lactic acid, pyruvic acid, lactate/pyruvate ratios, phosphocreatine, NADH(NADH+H⁺) or NADPH(NADPH+H⁺), NAD or NADP levels, ATP, anaerobic threshold, reduced coenzyme Q, oxidized coenzyme Q; total coenzyme Q, oxidized cytochrome C, reduced cytochrome C, oxidized cytochrome C/reduced cytochrome C ratio, acetoacetate, β-hydroxy butyrate, acetoacetate/β-hydroxy butyrate ratio, 8-hydroxy-2′-deoxyguanosine (8-OHdG), levels of reactive oxygen species, levels of oxygen consumption (VO2), levels of carbon dioxide output (VCO2), and respiratory quotient (VCO2/VO2).

12. The method of claim 1, wherein the administering results in increased thiol levels compared to levels before administration of the cysteamine or derivative thereof or cystamine or derivative thereof.

13. The method of claim 1, wherein the cysteamine or cystamine or derivative thereof is formulated in a tablet or capsule which is enterically coated.

14. The method of claim 1, wherein the cysteamine or derivative thereof or cystamine or derivative thereof is administered parenterally.

15. The method of claim 1, wherein the cysteamine or derivative thereof or cystamine or derivative thereof is administered orally.

16. The method of claim 1, wherein the cysteamine or derivative thereof or cystamine or derivative thereof further comprises a pharmaceutically acceptable carrier.

17. The method of claim 1, wherein the cysteamine or derivative thereof or cystamine or derivative thereof is formulated as a sterile pharmaceutical composition.

18. The method of claim 1, wherein the inherited mitochondrial disorder is selected from the group consisting of Friedreich's ataxia, Leber's hereditary optic neuropathy and Leigh's syndrome.

19. (canceled)

20. The method of claim 18, wherein the cysteamine or derivative thereof or cystamine or derivative thereof is administered topically in the eye.

21. (canceled)

22. The method of claim 1, wherein the cysteamine or derivative thereof or cystamine or derivative thereof is administered with a second agent useful to treat inherited or acquired mitochondrial diseases or disorders.

23. The method of claim 22, wherein the second agent is selected from the group consisting of coenzyme Q10, coenzyme Q10 analogs, idebenone, decylubiquinone, Epi-743, resveratrol and analogs thereof, arginine, vitamin E, tocopherol, MitoQ, glutathione peroxidase mimetics, levo-carnitine, acetyl-L-carnitine, dichloroacetate, dimethylglycine and lipoic acid.

24. The method of claim 1, wherein the subject is a child or adolescent.

25. The method of claim 1, wherein the administering results in improved results in the Newcastle Paediatric Mitochondrial Disease Scale and Barry Albright Dystonia Scale compared to levels before administration of the cysteamine or derivative thereof or cystamine or derivative thereof.
